(19) United States
(12) Patent Application Publication
(10) Pub. No.: US 2008/0065049 A1
(43) Pub. Date: Mar. 13, 2008

(54) VARIABLE STIFFNESS DIRECT INJECTION SYSTEM

(75) Inventors: Chad G. Harris, Albertville, MN (US); Timothy J. Mickley, Elk River, MN (US)

Correspondence Address: KENYON & KENYON LLP, 1500 K STREET N.W., SUITE 700, WASHINGTON, DC, 20005, US

(73) Assignee: BOSTON SCIENTIFIC SCIMED, INC.

(21) Appl. No.: 11/851,600

(22) Filed: Sep. 7, 2007

Related U.S. Application Data
(60) Provisional application No. 60/843,708, filed on Sep. 12, 2006.

(51) Int. Cl. A61M 25/01

(52) U.S. Cl. 604/525

(57) ABSTRACT

A direct injection system with a small diameter that can be easily placed at a desired treatment location is provided, where the system comprises an outer guide catheter having a predefined bend at the distal end. For example, the outer guide catheter may be no larger than a 7 Fr catheter. According to the invention, various mechanisms may be used to alter the curvature of the predefined bend in the catheter, allowing an operator to easily place the distal end of an injection catheter at a desired treatment location. Alternatively, the injection catheter itself has a predefined bend at the distal end, wherein the curvature can be adjusted by adjusting the position of the injection catheter within a guide catheter.

### RELATED APPLICATIONS

This application claim benefit of 60/843,708, filed Sep. 12, 2006, which is incorporated herein in its entirety.

### TECHNICAL FIELD

The present invention relates to medical devices, specifically devices for delivering a therapeutic agent or performing a procedure within the body of a patient.

### BACKGROUND

The delivery of therapeutic agents to diseased muscle or other tissue is an important, often repeated, procedure in the practice of modern medicine. Therapeutic agents, including therapeutic drugs and genetic material, may be used to treat, regenerate, or otherwise affect the muscle surface or the interior of the muscle itself. Such therapy can promote revascularization and create new formation of muscle, such as the myocardium of the heart. For example, many of the treatments for a failing heart due to congestive heart failure entail the delivery of therapeutic agents, growth factors, nucleic acids, gene transfection agents, or cellular transplants, e.g. fetal cardiomyocytes, allogeneic cardiomyocytes, allogeneic or autologous myocytes, and other potentially pluripotential cells from autologous or allogeneic bone marrow or stem cells.

Current methods for delivering therapeutic agents to muscle, such as the heart muscle, entail direct injection of a genetic cell or therapeutic drug into the muscle to be treated. Delivery of therapeutic agents has been proposed or achieved using medical devices such as catheters, needle devices and various coated implantable devices such as stents. The cells and agents can be injected directly or can be formulated into gels, sealants, or microparticles for injection.

During such treatments, an operator of a direct injection system may be attempting to treat a three-dimensional space, such as the chamber of a patient's heart. These regions may be treated using, for example, a catheter having a distal bend. In some applications, a catheter having a distal bend will utilize an interior tube having a similar bend. By manipulating the catheter and the interior tube relative to each other, an operator of the device may position the distal end at a desired treatment location.

However, such devices may be undesirable due to the relatively large catheter size required. For example, a typical configuration is to use a 9 Fr external catheter and a 7 Fr internal catheter, each having a predefined bend. By manipulating the two catheters relative to each other, a desired shape may be given to the distal end of the device. However, such large catheters require a respectively large opening in the body during treatment, which can make treatment of certain areas difficult or not possible and can prolong recovery time. In addition, use of the dual-bend systems may require substantial manipulation to position the distal end of the system at the desired treatment location. There is therefore a need for a direct injection system having a small diameter that an operator may easily position at a desired treatment location.

### SUMMARY OF THE INVENTION

The present invention relates to a direct injection system having a decreased diameter that is easily positioned at a desired treatment location by an operator of the device.

In an embodiment of the invention, a direct injection system with a small diameter is provided, wherein the system comprises an outer guide catheter having a predefined bend at the distal end. The outer guide catheter may be, for example, no larger than a 7 Fr catheter. A straightening element, such as a relatively stiff wire or tube, is positioned within the outer guide catheter and may be positioned along the longitudinal axis of the outer guide catheter by an operator of the device. In the case of a relatively stiff wire, a direct injection catheter may be positioned in the outer guide catheter adjacent to the wire. In the case of a relatively stiff tube, a direct injection catheter may be disposed within the relatively stiff tube. When an operator of the system moves the relatively stiff wire or tube toward the distal end of the outer guide catheter, the relatively stiff wire or tube causes the outer guide catheter to straighten. By changing the positioning of the relatively stiff wire or tube within the outer guide catheter, the amount of the bend can be controlled. Thus the distal end of the direct injection catheter may be positioned accurately at the desired treatment location. Once positioned, the direct injection catheter may be used to deliver a therapeutic agent.

In another embodiment of the invention, a direct injection system with a small diameter is provided, wherein the system comprises an outer guide catheter having a predefined bend at the distal end. The outer guide catheter may be, for example, no larger than a 7 Fr catheter. A direct injection catheter is disposed within the outer guide catheter, and may be moved along the longitudinal axis of the outer guide catheter by an operator of the device. The distal end of the direct injection catheter comprises a shaft with regions of varying stiffness, where the distal-most region of the shaft may be the most flexible, and a region or regions toward the proximal end of the tube may be less flexible. When the most flexible region of the shaft is placed within the predefined bend in the outer guide catheter, the bend is not substantially altered. When an operator positions a less-flexible region of the shaft within the bend, the bend is straightened. If an operator positions the least flexible portion of the tube within the bend, the outer guide catheter is straightened to a maximum amount for the system. Thus the operator may control the shape of the outer guide catheter to position the distal end of the direct injection catheter at a desired treatment location. As an alternative to regions of varying stiffness along the distal end of the shaft of the direct injection catheter, the stiffness along the distal end of the shaft can vary continuously along the length of the shaft.

In another embodiment of the invention, a direct injection system with a small diameter is provided, wherein the device comprises an outer guide catheter and an injection catheter. The outer guide catheter may be no larger than a 7 Fr catheter. The injection catheter has a predefined bent shape, for example by a casing made of a shape-memory. The outer guide catheter forces the injection catheter into a straightened configuration. When an operator of the device extends the injection catheter from the distal end of outer guide catheter, it resumes part or all of the predefined bent shape. The degree of curvature may be controlled by an operator based on the length of the injection catheter that is extended. The operator may then place the distal end of the injection catheter at the desired treatment location.

### BRIEF DESCRIPTION OF THE DRAWINGS

FIGS. 1A-1C show side cut-away views of a direct injection system having a small outer guide catheter with a predefined bend and a relatively stiff tube.

FIGS. 2A-2E show side cut-away views of a direct injection system with a direct injection catheter having a variable flexibility at the distal end.

FIGS. 3A-3B show side cut-away views of a direct injection system having an injection catheter comprising a predefined bent shape.

FIGS. 4A-4B show side cut-away views of a direct injection system positioned at a desired treatment location.

### DETAILED DESCRIPTION

A direct injection system is provided, which comprises a small-diameter catheter. In some embodiments, the outer guide catheter has a predefined bend at the distal end, and the device includes a mechanism an operator may use to adjust the degree of curvature of the outer guide catheter. In some embodiments, an injection catheter disposed within the small-diameter outer guide catheter has a predefined bend, and the outer guide catheter may be used to adjust the degree of curvature of the bend. The invention will now be described with reference to the drawings, in which like reference numerals are used to designate like structures throughout.

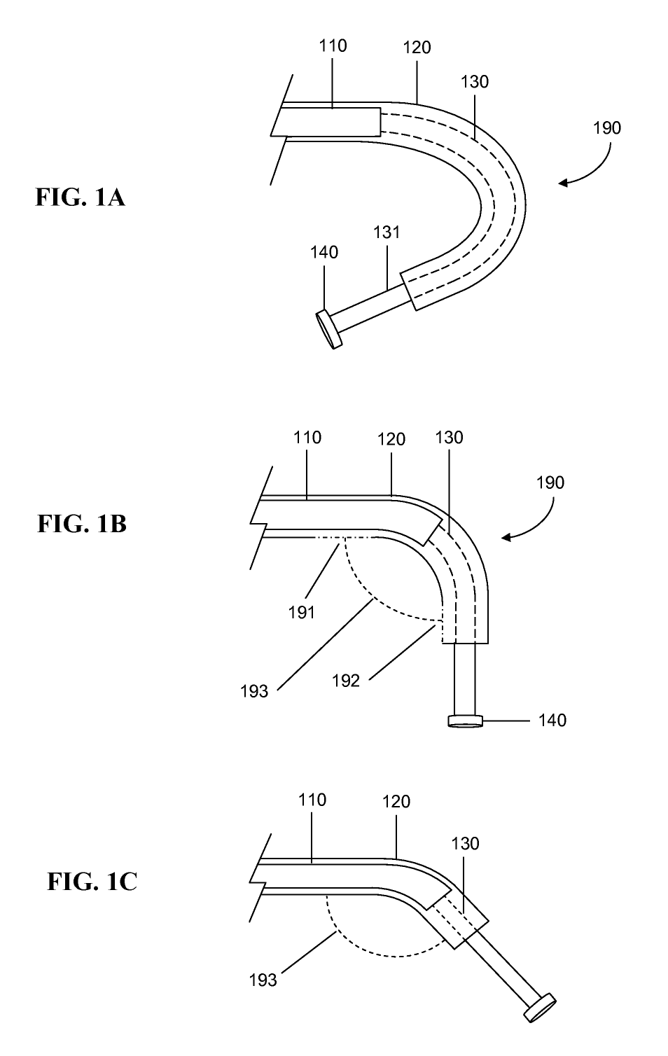

FIG. 1 shows an embodiment of the invention having a small outer guide catheter with a predefined bend and a straightening element. An outer guide catheter 120 has a predefined bend 190 near the distal end of the catheter. A straightening element 110 is disposed within the outer guide catheter 120. The straightening element 110 may be, for example, a relatively stiff wire disposed within and along the interior surface of the outer guide catheter 120, or a relatively stiff tube as shown in FIG. 1. An injection catheter 130 is disposed within the relatively stiff tube 110. The injection catheter 130 may be positioned along the longitudinal axis of the outer guide catheter 120, such that the delivery point 140 extends past the end of the catheter 120.

FIG. 1A shows the system with the relatively stiff tube 110 positioned away from the distal end of the outer guide catheter 120. The bend of the outer guide catheter 120 is thus at its maximum curvature. To decrease the curvature, i.e., straighten the outer guide catheter 120, an operator may move the relatively stiff tube 110 toward the distal end of the outer guide catheter 120.

FIG. 1B shows the same system as shown in FIG. 1A, with the relatively stiff tube 110 positioned further toward the distal end of the catheter 120 than in FIG. 1A. When placed in such a configuration, the stiff tube 110 decreases the curvature of the outer guide catheter 120. As will be understood by one of skill in the art, configurations other than those shown in FIGS. 1A-1B are possible. For example, the relatively stiff tube 110 may be placed within the outer guide catheter 120 such that the curvature of the outer guide catheter is less than the curvature shown in FIG. 1A, but greater than that shown in FIG. 1B.

FIG. 1C shows the direct injection system with the relatively stiff tube 110 positioned closer to the distal end of the outer guide catheter 120 than shown in FIGS. 1A-1B. The outer guide catheter 120 is therefore in a configuration where the bend has the least curvature. In some embodiments, this configuration will result in the catheter having no curvature, i.e., completely straightened. In some embodiments, the catheter may have a minimum curvature, i.e., the bend may only be straightened to a certain point, after which it may not be straightened further.

As used herein, curvature may be measured relative to the angle between two straight sections of the outer guide catheter, where one section is closer to the distal end of the catheter than the bend, and the other section is closer to the proximal end of the catheter than the bend. For example, the curvature of the configuration shown in FIG. 1B may be described with respect to the angle between two sections of the outer guide catheter 191, 192, where the sections are on opposite sides of the bend 190. An arc 193 is shown across the angle for reference. In the example configuration, the curvature corresponds to an angle of roughly 90°. In some embodiments, the outer guide catheter may be adjusted to have a curvature corresponding to an angle between 0° (maximum curvature) and 180° (minimum curvature; i.e., the catheter is straight).

By adjusting the curvature of the bend in the outer guide catheter 120, an operator of the device may position the distal end of the injection catheter 140 at an intended treatment location. For example, if a desired treatment site is on the side of the interior of the left ventricle of a patient's heart, the distal end of the device may be positioned within the left ventricle of the heart. The operator may then position the relatively stiff wire or tube 110 so as to adjust the curvature of the bend in the outer guide catheter. When the bend is at a desired curvature, the distal end of the injection catheter 140 may be extended to contact the desired treatment site.

The system as described allows the use of an outer guide catheter 120 having a relatively small diameter. Due to the small size of the outer guide catheter 120, the system can reach target areas more easily, and the procedure does not require a large opening in the patient's body. For example, in some embodiments the outer guide catheter is not larger than a 7 Fr catheter. A “7 Fr” catheter is a typical gauge of catheter, where a 3 Fr catheter has an outer diameter of 1 mm. A 7 Fr catheter therefore has an outer diameter of approximately 2.3333 mm.

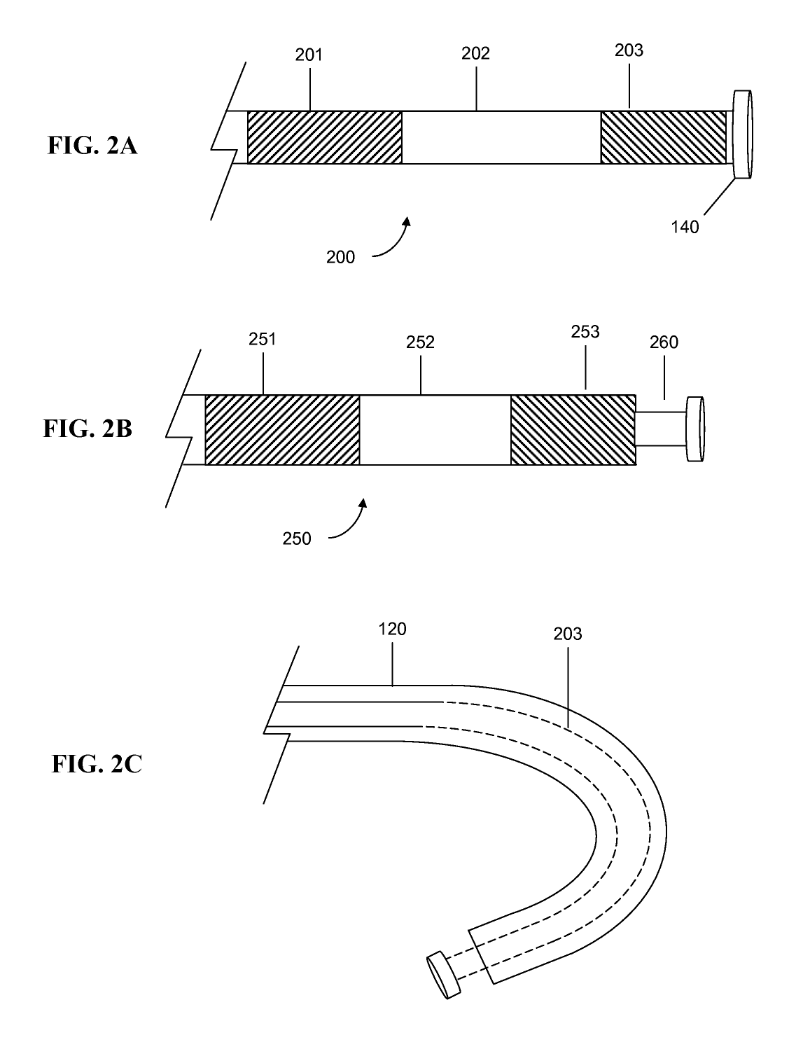

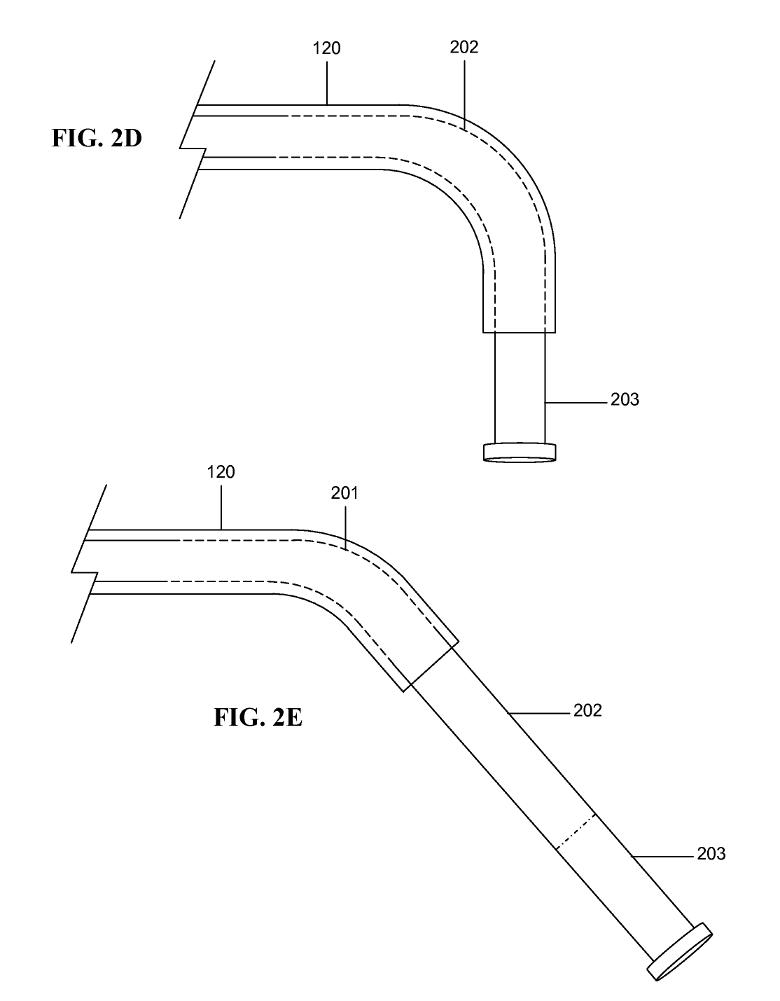

FIG. 2 shows an embodiment of the invention having a variable flexibility injection catheter. FIG. 2A shows the injection catheter. In some embodiments, the injection catheter 200 has multiple regions 201, 202, 203, where each region has a different flexibility. Three regions are shown in FIG. 2A; in various embodiments more or fewer regions may be used. Each region may be made of a different material, or each region may be comprised of a shaft or tube having a different thickness. Region 203 is more flexible, i.e., more easily bent, than regions 201 and 202. Similarly, region 201 is less flexible, i.e., less easily bent, than regions 202 and 203. Region 202 is of medium flexibility, i.e., more flexible than region 201, but less flexible than region 203. In some embodiments, the injection catheter may have a completely variable flexibility near the distal end. That is, the flexibility may change continuously near the distal end of the injection catheter. In this embodiment, the area closest to the distal end is the most flexible, and the flexibility of the injection catheter decreases in proportion to the distance from the distal tip of the outer guide catheter. Such a catheter may be referred to as having a “gradient” flexibility.

In some embodiments, the injection catheter may have a uniform flexibility throughout some or all of its length, specifically near the distal end. A cylindrical sheath may be disposed around the injection catheter. In such embodiments, the sheath may have variable flexibility. FIG. 2B shows an injection catheter 260 disposed within a variable-flexibility sheath 250. Similar to the injection catheter described with respect to FIG. 2A, the sheath may have multiple regions 251, 252, 253, with each region having a different flexibility. As with the variable-flexibility injection catheter previously described, the sheath is more flexible in regions closer to the distal end, i.e., region 253, and less flexible in regions farther from the distal end, i.e., region 251. The sheath may also have a gradient flexibility.

FIG. 2C shows a direct injection system that includes an injection catheter having a variable flexibility near the distal end, which is disposed inside a small-diameter outer guide catheter having a predefined bend. In some embodiments, the outer guide catheter is a 7 Fr or smaller catheter. An operator of the device may position the injection catheter within the outer guide catheter, such that a specific portion of the injection catheter is disposed within the predefined bend in the outer guide catheter. When the injection catheter is placed such that the most flexible portion of the catheter 203, i.e., the portion closest to the distal end, is within the bend of the outer guide catheter, the bend of the outer guide catheter is at a maximum curvature. An operator may move the injection catheter through the outer guide catheter, in the distal direction. When a less-flexible portion of the injection catheter is placed within the predefined bend of the outer guide catheter, the curvature of the bend may be decreased. FIG. 2D shows the direct injection system of FIG. 2C, where a less-flexible portion 202 of the injection catheter is disposed within the bend of the outer guide catheter 120. When the less-flexible portion is disposed within the bend, the outer guide catheter is straightened to a position having a lower curvature than in the configuration shown in FIG. 2C. It will be understood that when the injection catheter is positioned at a location between those shown in FIGS. 2C and 2D, the bend may have a curvature between those shown in FIGS. 2C and 2D. For example, if the injection catheter has a gradient flexibility, any curvature between those shown may be achieved. The injection catheter may also be positioned farther toward the distal end of the outer guide catheter, as shown in FIG. 2E, such that the least-flexible region of the injection catheter 201 is disposed within the predefined bend. In such a configuration, the bend in the outer guide catheter 120 is straightened to a minimum curvature. In some embodiments, when the least-flexible portion of the injection catheter is placed in the predefined bend, the outer guide catheter may be completely straightened, i.e., it may have no curvature. In some embodiments, the outer guide catheter may be adjusted to have a curvature corresponding to an angle between 0° and 180°.

The configurations shown in FIGS. 2C-2E are shown and described with respect to a variable-flexibility injection catheter as shown in FIG. 2A. Similar configurations may be achieved using the variable-flexibility sheath shown in FIG. 2B. In such configurations the sheath may be positioned as described with respect to the injection catheter in FIGS. 2C-2E to achieve a desired curvature of the outer guide catheter. The injection catheter may then be positioned independently of the sheath and the outer guide catheter. This may be used, for example, where a curvature as shown in FIG. 2D is desired, but the treatment site is located relatively far from the distal tip of the outer guide catheter. Such a configuration allows the injection catheter to be extended the necessary amount past the distal tip of the outer guide catheter, without altering the curvature of the outer guide catheter. The curvature of the bend in the outer guide catheter is formed and held in place by the sheath, which allows an operator to extend the injection catheter without altering the curvature of the bend.

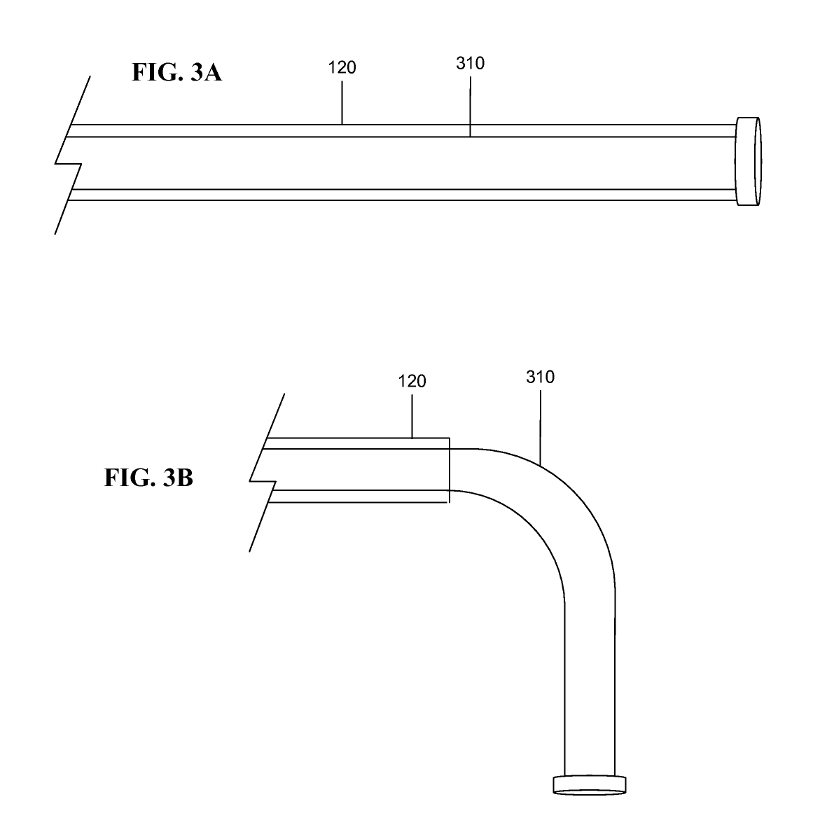

In another embodiment of the invention, shown in FIG. 3, the injection catheter comprises or is enclosed in a sheath comprising a shape-memory material. For example, the injection catheter may comprise or be enclosed in a sheath comprising Nitinol. The injection catheter or sheath may be constructed such that it has an initial curved shape. The injection catheter and sheath, if present, are disposed within an outer guide catheter 120. In some embodiments, the outer guide catheter 120 is a 7 Fr or smaller catheter. When placed within the outer guide catheter 120, the injection catheter 310 assumes the shape of the outer guide catheter. The injection catheter 310 may be disposed within the outer guide catheter 120 such that a portion of the injection catheter protrudes from the distal tip of the outer guide catheter. Any such protruding section, if comprised of or encased in a sheath comprising a shape memory material, will return to the initial shape.

FIG. 3A shows the injection catheter 310 disposed fully within the outer guide catheter 120. FIG. 3B shows the injection catheter 310 protruding from the outer guide catheter 120. The portion of the injection catheter protruding from the outer guide catheter assumes the shape defined by the shape-memory material as previously described. By adjusting the amount of the injection catheter that protrudes from the outer guide catheter, an operator may achieve a desired curvature of the injection catheter. In some embodiments, the outer guide catheter may be adjusted to have a curvature corresponding to an angle between 0° and 180°. The injection catheter may be made with a predefined bend using other materials, such as an elastic metal or a resilient plastic. When withdrawn into a relatively straight outer guide catheter, the bent catheter straightens. When extended, the bend catheter returns to its bent configuration, where the degree of curvature and general shape of the bend may depend on the amount of extension. Any such material, that can be given an initial predefined shape to which the material may return when subjected to or released from the appropriate stress, may be referred to as a shape-retaining material. Shape-memory materials such as Nitinol, resilient plastics, braided-metal sheets, and elastic metals are non-limiting examples of shape-retaining materials.

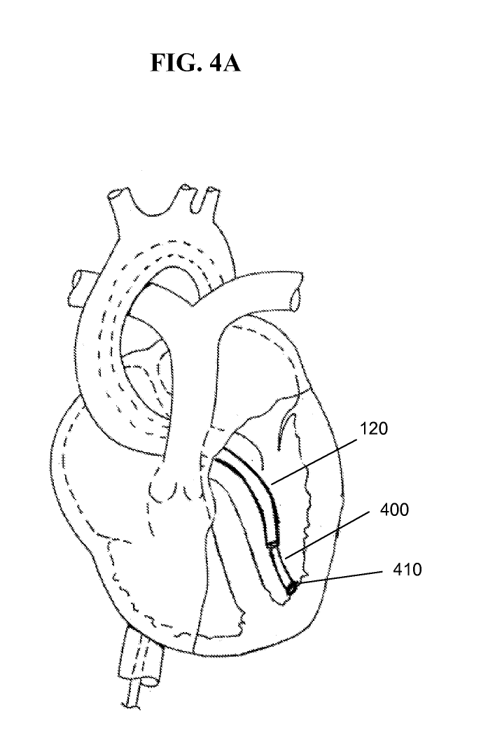

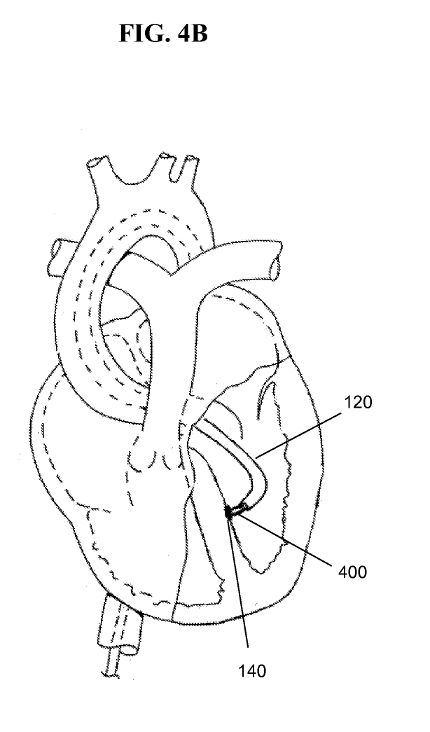

FIG. 4 shows systems according to the present invention positioned at a desired treatment location within the left ventricle of a patient's heart. FIG. 4A shows an exemplary arrangement of an outer guide catheter 120, with an injection catheter 400 positioned to deliver a therapeutic agent to a desired treatment site 410. FIG. 4B shows an exemplary arrangement of an outer guide catheter 120 having a predefined curve. An injection catheter 400 is extended from the outer guide catheter to contact the desired treatment site 410. The outer guide catheter 120 and injection catheter 400 in FIGS. 4A-4B may be in any of the configurations described with respect to FIGS. 1-3. In each arrangement, the curve of the outer guide catheter 120 and/or injection catheter 400 may be adjusted to position the distal end of the injection catheter at the desired treatment site.

The various arrangements and combinations of structure described with respect to each figure may be used in combinations other than those described. For example, various embodiments of the invention may incorporate one or more sheaths around the injection catheter. Devices according to the present invention may be used as part of or in conjunction with other direct injection systems and devices, such as those described in U.S. Pat. Nos. 6,238,406, 6,767,338, and 6,939,322. Various devices and structures may be used to deliver therapeutic agents, for example by incorporating different treatment devices into the distal end of the injection catheter. Examples of such structures are described in U.S. application Ser. No. 10/121,618, filed Apr. 15, 2002.

Although the present invention has been described with respect to specific treatment locations, it may be adapted and/or utilized to treat various other locations within the body of a patient.

The term “therapeutic agent” as used throughout includes one or more “therapeutic drugs” or “genetic material.” The term “therapeutic agent” used herein includes pharmaceutically active compounds, nucleic acids with and without carrier vectors such as lipids, compacting agents (such as histones), virus (such as adenovirus, adenoassociated virus, retrovirus, lentivirus and a-virus), polymers, hyaluronic acid, proteins, cells and the like, with or without targeting sequences. The therapeutics administered in accordance with the invention includes the therapeutic agent(s) and solutions thereof. The therapeutic agent may be any pharmaceutically acceptable agent such as a non-genetic therapeutic agent, a biomolecule, a small molecule, or cells.

Exemplary non-genetic therapeutic agents include anti-thrombogenic agents such heparin, heparin derivatives, prostaglandin (including micellar prostaglandin E1), urokinase, and PPack (dextrophenylalanine proline arginine chloromethylketone); anti-proliferative agents such as enoxaprin, angiopeptin, sirolimus (rapamycin), tacrolimus, everolimus, zotarolimus, monoclonal antibodies capable of blocking smooth muscle cell proliferation, hirudin, and acetylsalicylic acid; anti-inflammatory agents such as dexamethasone, rosiglitazone, prednisolone, corticosterone, budesonide, estrogen, estrodiol, sulfasalazine, acetylsalicylic acid, mycophenolic acid, and mesalamine; anti-neoplastic/anti-proliferative/anti-mitotic agents such as paclitaxel, epothilone, cladribine, 5-fluorouracil, methotrexate, doxorubicin, daunorubicin, cyclosporine, cisplatin, vinblastine, vincristine, epothilones, endostatin, trapidil, halofuginone, and angiostatin; anti-cancer agents such as antisense inhibitors of c-myc oncogene; anti-microbial agents such as triclosan, cephalosporins, aminoglycosides, nitrofurantoin, silver ions, compounds, or salts; biofilm synthesis inhibitors such as non-steroidal anti-inflammatory agents and chelating agents such as ethylenediaminetetraacetic acid, O,O′-bis(2-aminoethyl)ethyleneglycol-N,N,N′,N′-tetraacetic acid and mixtures thereof; antibiotics such as gentamycin, rifampin, minocyclin, and ciprofolxacin; antibodies including chimeric antibodies and antibody fragments; anesthetic agents such as lidocaine, bupivacaine, and ropivacaine; nitric oxide; nitric oxide (NO) donors such as linsidomine, molsidomine, L-arginine, NO-carbohydrate adducts, polymeric or oligomeric NO adducts; anti-coagulants such as D-Phe-Pro-Arg chloromethyl ketone, an RGD peptide-containing compound, heparin, antithrombin compounds, platelet receptor antagonists, anti-thrombin antibodies, anti-platelet receptor antibodies, enoxaparin, hirudin, warfarin sodium, Dicumarol, aspirin, prostaglandin inhibitors, platelet aggregation inhibitors such as cilostazol and tick antiplatelet factors; vascular cell growth promotors such as growth factors, transcriptional activators, and translational promotors; vascular cell growth inhibitors such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; cholesterol-lowering agents; vasodilating agents; agents which interfere with endogenous vascoactive mechanisms; inhibitors of heat shock proteins such as geldanamycin; angiotensin converting enzyme (ACE) inhibitors; beta-blockers; bAR kinase (bARKct) inhibitors; phospholamban inhibitors; protein-bound particle drugs such as ABRAXANE™; and any combinations and prodrugs of the above.

Exemplary biomolecules include peptides, polypeptides and proteins; oligonucleotides; nucleic acids such as double or single stranded DNA (including naked and cDNA), RNA, antisense nucleic acids such as antisense DNA and RNA, small interfering RNA (siRNA), and ribozymes; genes; carbohydrates; angiogenic factors including growth factors; cell cycle inhibitors; and anti-restenosis agents. Nucleic acids may be incorporated into delivery systems such as, for example, vectors (including viral vectors), plasmids or liposomes.

Non-limiting examples of proteins include serca-2 protein, monocyte chemoattractant proteins (“MCP-1) and bone morphogenic proteins (“BMP's”), such as, for example, BMP-2, BMP-3, BMP-4, BMP-5, BMP-6 (Vgr-1), BMP-7 (OP-1), BMP-8, BMP-9, BMP-10, BMP-11, BMP-12, BMP-13, BMP-14, BMP-15. Preferred BMPS are any of BMP-2, BMP-3, BMP-4, BMP-5, BMP-6, and BMP-7. These BMPs can be provided as homdimers, heterodimers, or combinations thereof, alone or together with other molecules. Alternatively, or in addition, molecules capable of inducing an upstream or downstream effect of a BMP can be provided. Such molecules include any of the “hedghog” proteins, or the DNA's encoding them. Non-limiting examples of genes include survival genes that protect against cell death, such as anti-apoptotic Bcl-2 family factors and Akt kinase; serca 2 gene; and combinations thereof Non-limiting examples of angiogenic factors include acidic and basic fibroblast growth factors, vascular endothelial growth factor, epidermal growth factor, transforming growth factor ÿ and ÿ, platelet-derived endothelial growth factor, platelet-derived growth factor, tumor necrosis factor ÿ, hepatocyte growth factor, and insulin like growth factor. A non-limiting example of a cell cycle inhibitor is a cathespin D (CD) inhibitor. Non-limiting examples of anti-restenosis agents include p15, p16, p18, p19, p21, p27, p53, p57, Rb, nFkB and E2F decoys, thymidine kinase (“TK”) and combinations thereof and other agents useful for interfering with cell proliferation.

Exemplary small molecules include hormones, nucleotides, amino acids, sugars, and lipids and compounds have a molecular weight of less than 100 kD.

Exemplary cells include stem cells, progenitor cells, endothelial cells, adult cardiomyocytes, and smooth muscle cells. Cells can be of human origin (autologous or allogenic) or from an animal source (xenogenic), or genetically engineered. Non-limiting examples of cells include side population (SP) cells, lineage negative (Lin−) cells including Lin-CD34−, Lin-CD34+, Lin-cKit+, mesenchymal stem cells including mesenchymal stem cells with 5-aza, cord blood cells, cardiac or other tissue derived stem cells, whole bone marrow, bone marrow mononuclear cells, endothelial progenitor cells, skeletal myoblasts or satellite cells, muscle derived cells, go cells, endothelial cells, adult cardiomyocytes, fibroblasts, smooth muscle cells, adult cardiac fibroblasts+5-aza, genetically modified cells, tissue engineered grafts, MyoD scar fibroblasts, pacing cells, embryonic stem cell clones, embryonic stem cells, fetal or neonatal cells, immunologically masked cells, and teratoma derived cells.

Any of the therapeutic agents may be combined to the extent such combination is biologically compatible.

Systems and devices as used with the present invention may also contain a radio-opacifying agent within its structure to facilitate viewing the medical device during insertion and at any point while the device is implanted. Non-limiting examples of radio-opacifying agents are bismuth subcarbonate, bismuth oxychloride, bismuth trioxide, barium sulfate, tungsten, and mixtures thereof.

The examples described and illustrated herein are merely illustrative, as numerous other embodiments may be implemented without departing from the spirit and scope of the present invention. Moreover, while certain features of the invention may be shown on only certain embodiments or configurations, these features may be exchanged, added, and removed from and between the various embodiments or configurations while remaining within the scope of the invention. Likewise, methods described and disclosed may also be performed in various sequences, with some or all of the disclosed steps being performed in a different order than described while still remaining within the spirit and scope of the present invention.

What is claimed is:

1. A system for guiding an injection catheter to a target area, the system comprising:
an outer guide catheter having a proximal end and a distal end, the outer guide catheter having a predefined bend near the distal end;
an injection catheter disposed within and axially aligned with the outer guide catheter; and
a straightening element disposed within and axially aligned with the outer guide catheter, the straightening element moveable from a first position to a second position in the region of the predefined bend of the outer guide catheter, the straightening element being closer to the distal end of the outer guide catheter when it is in the second position;
wherein the curvature of the bend is greater when the straightening element is in the first position than when the straightening element is in the second position.

2. The system of claim 1 wherein the outer guide catheter is not larger than about a 7 Fr catheter.

3. The system of claim 1 wherein the straightening element is a stiff rod, and the injection catheter is adjacent to the rod.

4. The system of claim 1 wherein the straightening element is a relatively stiff tube, and the injection catheter is disposed within and axially aligned with the relatively stiff tube.

5. The system of claim 4 wherein the curvature of the bend has a minimum curvature when the relatively stiff tube is in the second position and a maximum curvature when the relatively stiff tube is in the first position.

6. The system of claim 5 wherein the minimum curvature corresponds to an angle of about 180° and the maximum curvature corresponds to an angle of about 0°.

7. A system for guiding an injection catheter to a target area, the system comprising:
an outer guide catheter having a proximal end and a distal end, the outer guide catheter having a predefined bend near the distal end; and
a straightening element having a distal end, disposed within and axially aligned with the outer guide catheter, wherein the straightening element is more flexible near the distal end and less flexible in an area proximal to the distal end;
wherein the straightening element is moveable from a first position to a second position in the region of the predefined bend of the outer guide catheter, the straightening element being closer to the distal end of the outer guide catheter when it is in the second position; and
wherein the curvature of the bend is greater when the straightening element is in the first position than when the straightening element is in the second position.

8. The system of claim 7 wherein the straightening element is part of an injection catheter.

9. The system of claim 7 wherein the straightening element is a sheath, and wherein the device further comprises an injection catheter disposed within and axially aligned with the sheath.

10. The system of claim 7 wherein the outer guide catheter is not larger than about a 7 Fr catheter.

11. The system of claim 7 wherein the curvature of the bend has a minimum curvature when the straightening element is in the second position and a maximum curvature when the straightening element is in the first position.

12. The system of claim 11 wherein the minimum curvature corresponds to an angle of about 180° and the maximum curvature corresponds to an angle of about 0°.

13. The system of claim 7 wherein the straightening element has a gradient flexibility.

14. The system of claim 7 wherein the straightening element comprises a plurality of regions, and wherein each region has a different degree of flexibility.

15. The system of claim 7 wherein the straightening element has a non-uniform thickness.

16. A system for guiding an injection catheter to a target area, the system comprising:
an outer guide catheter having a proximal end and a distal end; and
an injection catheter disposed within and axially aligned with the outer guide catheter, the injection catheter having a distal shaft comprising a shape-retaining material;
wherein the distal shaft has a bent shape when in a relaxed condition;
wherein the injection catheter is moveable from a first position to a second position;
wherein the distal shaft of the injection catheter protrudes from the distal end of the outer guide catheter when the injection catheter is in the second position such that the distal shaft is in the relaxed bent shape; and
wherein the distal shaft of the injection catheter is withdrawn at least partially into the outer guide catheter in the first position to straighten the distal shaft relative to its relaxed bent shape.

17. The system of claim 16, wherein the shape-retaining material is a shape-memory material.

18. The system of claim 16, further comprising a sheath disposed around and axially aligned with the injection catheter.

19. The system of claim 16 wherein the curvature of the distal shaft is proportional to the distance between the first position and the second position.

20. The system of claim 19 wherein the distal shaft has a minimum curvature corresponding to an angle of about 180° and a maximum curvature corresponding to an angle of about 0°.

21. The system 16 wherein the outer guide catheter is not larger than about a 7 Fr catheter.
